(19) United States
(12) Patent Application Publication
(10) Pub. No.: US 2011/0040372 A1
(43) Pub. Date: Feb. 17, 2011

(54) Coated Endoluminal Implant

(76) Inventors: Palle M. Hansen, Bjaeverskov (DK); Jesper Thyregod, Roskilde (DK)

Correspondence Address: BRINKS HOFER GILSON & LIONE/CHICAGO/COOK, PO BOX 10395, CHICAGO, IL, 60610, US

(21) Appl. No.: 12/918,298

(22) PCT Filed: Feb. 13, 2009
(86) PCT No.: PCT/US09/00935

§ 371 Date: Oct. 7, 2010

Related U.S. Application Data
(60) Provisional application No. 61/066,236, filed on Feb. 19, 2008.

(51) Int. Cl. A61F 2/82; B05D 1/02

(52) U.S. Cl. 623/1.46; 118/665; 427/2.25

(57) ABSTRACT

A stent structure (30) particularly for use in treating cerebral aneurysms is provided with a fibrous coating (34) which reduces the permeability of the underlying stent (32) to an extent that the flow through the stent structure is sufficiently suppressed to allow for resorbtion of an aneurysm while still providing sufficient flow through the stent structure to allow for flow into the perforator vessels. The fibrous coating may have different permeabilities in different zones of the underlying stent (32). The coating could be applied to other devices such as filters and occluders.

This application is a continuation of PCT/U.S. 2009/000935,filed Feb. 13, 2009, which claims priority to U.S. Provisional Application No. 61/066,236, filed Feb. 19, 2008, both of which are hereby incorporated by reference herein.

### TECHNICAL FIELD

The present invention relates to a coated endoluminal implant and to a method of manufacturing such an implant. The preferred embodiment is directed to a coated stent for deployment in a cerebral artery for the treatment of aneurysms.

### BACKGROUND ART

In the context of treatment of aneurysms, recent studies have concluded that neuroendovascular procedures are safer and more convenient than surgical repair for a large number of medical cases. For general arterial aneurysms, it is known to locate a stent graft over the aneurysm so as to block the passage of blood into the aneurysm. Over time, as a result of the lack of blood pressure impinging on the internal walls of the aneurysm, this resorbs.

Recent studies have suggested that a reduction in fluid pressure into an aneurysm or a change in the nature of the flow which reduce the pressure on the internal walls of the aneurysm, are sufficient to repair the aneurysm. Recent studies by the applicant suggest that increasing stent strut size and segment length can change the porosity of the stent thus affecting the flow of blood to the aneurysm. U.S.-2007/0162104 discloses a specific braided structure devoid of any cover whatsoever and intended as a replacement to a conventional stent. The braided structure provides a mat with specific permeability and wire configurations intended to alter the haemodynamic qualities of the flow therethrough to a laminar flow which has a lower impact on an aneurysm.

More recently, there have been proposed endovascular procedures to treat cerebral aneurysms. It is estimated that around 2 to 3% of the population may be living with an unruptured cerebral aneurysm. However, cerebral aneurysms present particular challenges in light of the fact that the cerebral arteries are much more delicate and that they have large numbers of perforator vessels which must not be starved of blood.

WO-2007/051179 and WO-2001/87184 disclose cerebral stent structures which are provided with a partial occlusion covering sized and located on the stent so as to overlie the neck of an aneurism to as to block blood flow thereto. The remainder of the stent structures are no covered, such that blood flow to perforator vessels covered by the stent is not adversely affected. It will be apparent that such a solution requires careful design, positioning and orientation of the stent structure so ensure that only the aneurysm neck is occluded and not any adjacent perforator vessels.

U.S.-2004/0051201 discloses a method of providing a fabric or fabric-like covering suitable for a medical device and industrial filtration applications. The method involves electro spinning a coating onto a substrate, for example a stent, which coating is formed of fine polymer fibres which adhere to the surface of the substrate. The coating may be adapted for the delivery of a drug when applied to a stent.

### DISCLOSURE OF THE INVENTION

The present invention seeks to provide an improved coated endoluminal implant and improved method of manufacturing such an implant. The implant may be a stent, a vena cava filter, an occlusion device or any other of a variety of such endoluminally deployable implants known in the art. The invention also seeks to provide an improved coated stent for deployment in a cerebral artery for the treatment of aneurysms.

According to an aspect of the present invention, there is provided an endoluminally implantable cranial stent assembly for the treatment of cerebral aneurysms, including a stent in the form of a substantially open framework and a permeable covering of randomly oriented fibrous material, wherein the covering has a permeability able to reduce flow by between 30 to 50 percent of the flow through the stent.

Advantageously, the flow reduction is 40% or greater, most preferably around 40%.

Such reductions in flow have the advantage being able to provide treatment for an aneurysm by applying sufficient reduction of flow pressure within the aneurysm, as well as allowing sufficient blood blow into adjacent perforator vessels which the structure overlies. Moreover, the thickness of the fibre layer required to provide such a flow reduction can still be of a low profile to be suitable for cerebral vessels.

Thus, the preferred embodiments provide a covering which has sufficiently reduced permeability to provide for resorbance of an aneurysm while providing sufficient flow of blood to any perforator vessels within the area of coverage of the device or assembly. The latter effect is assisted by the suction force produced within the perforator vessels tending to draw blood thereinto.

Furthermore, the provision of such a covering does not substantially affect the flexibility of the stent, which allows it to be suitable for cerebral applications and any other applications which require a very flexible stent. Moreover, the fibrous covering can be thin, therefore avoiding adding excessively to the expanded diameter of the underlying stent.

According to another aspect of the present invention, there is provided an endoluminally implantable medical device including a substrate in the form of a substantially open framework and a permeable covering of randomly oriented fibrous material, wherein the covering has different permeabilities in different areas of the substrate.

According to another aspect of the present invention, there is provided an endoluminally implantable medical device including a substrate in the form of a substantially open framework and a permeable partial covering of randomly oriented fibrous material located on a part of the substrate.

According to another aspect of the present invention, there is provided a cerebral stent structure for the treatment of cerebral aneurysms, including a stent in the form of a substantially open framework and a covering of randomly oriented fibrous polymer material, wherein the covering is at least partially permeable.

According to another aspect of the present invention, there is provided a vena cava filter including a substrate formed as a substantially open framework and a covering of randomly oriented fibrous material over the framework, wherein the covering is at least partially permeable.

According to another aspect of the present invention, there is provided an endoluminal occlusion device including a substrate in the form of a substantially open framework and an occlusion barrier formed of randomly oriented fibrous material.

In all of the above aspects, it is preferable that the coating is an electro spun coating which adheres to the substrate by application of the fibres before they dry during electro spinning.

In all of the above aspects and embodiments of the invention, the covering may be of a polymer. It is also envisaged that the covering may be of a biologically active fibrous material, such as collagen or SIS. It is not excluded that a combination of a polymer and a biologically active fibrous material could be used in some embodiments.

According to another aspect of the present invention, there is provided a system for providing a coated endoluminal medical device including:

a substrate holder for holding a substrate in the form of a substantially open framework;

a nozzle arrangement direct able to the substrate holder;

a source of fluid fibrous material;

a pumping device operable to pump fluid fibrous material through the nozzle arrangement; and

a control unit operable to control the pumping device and the nozzle arrangement;

wherein the control unit is able to apply a variable coating to a substrate.

The source of fluid fibrous material may be a source of fluid polymeric material and/or a source of fluid biologically active material.

According to another aspect of the present invention, there is provided a method of coating endoluminal medical device including the steps of:

holding a substrate in the form of a substantially open framework on a substrate holder;

providing a nozzle arrangement directed at the substrate holder;

pumping fluid fibrous material through the nozzle arrangement; and

wherein the pumping arrangement applies a variable coating to the substrate.

### BRIEF DESCRIPTION OF THE DRAWINGS

Embodiments of the present invention are described below, by way of example only, with reference to and as illustrated in the accompanying drawings, in which:

FIG. 1 is a photograph of an example of an internal cartoid artery having two aneurysms therein;

FIG. 2 is a schematic diagram of a cartoid aneurysm of the type seen in FIG. 1;

FIG. 3 is a perspective view of an embodiment of coated stent;

FIG. 4 is a perspective view of another embodiment of coated stent;

FIG. 5 is a perspective view of an embodiment of partially coated stent;

FIG. 6 is a photograph of a spun coated fibre covering; and

FIG. 7 is a schematic diagram of an embodiment of electro spinning apparatus suitable for producing the stent and other coated devices disclosed herein.

### DESCRIPTION OF THE PREFERRED EMBODIMENTS

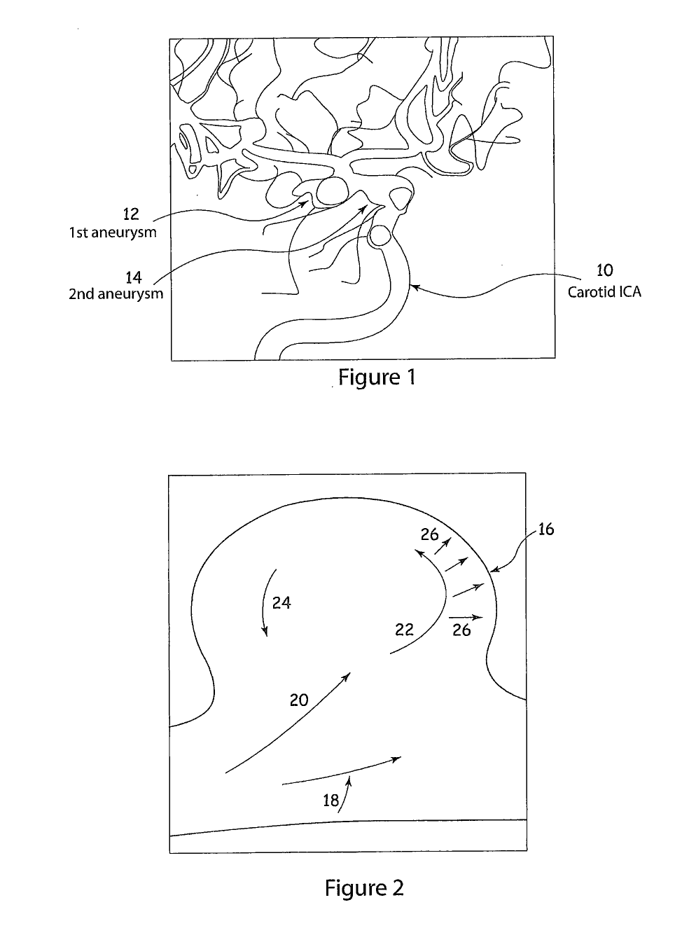

Referring to FIG. 1, this shows a picture of an internal cartoid artery 10 which has first and second unruptured aneurysms 12 and 14, respectively. Typically, such aneurysms are caused by a weakening of the artery walls in the region of the aneurysm, which causes a bulging at the location of weakness as a result of blood pressure. As the bulge expands, there is generated a flow of blood within the bulge or aneurysm itself, which causes its further expansion, typically until the point of rupture.

FIG. 2 shows in enlarged form a simulation of a cartoid aneurysm 16. The arrows 18-24 depict the flow of blood through the cartoid artery at the site of the aneurysm and in particular the turbulent flow into the aneurysm itself. This flow, particularly at the wall of the aneurysm opposite the direction of blood flow, applies pressure on the aneurysm, as shown by the arrows 26, tending to expand the aneurysm over time. In the case of a cerebral aneurysm, rupture typically results in a stroke and possible fatality. As explained above, it is believed that between 2 to 3% of the population suffers from cerebral aneurysms.

Referring again to FIG. 1, it can be seen that there are a great many perforator vessels coupled to the internal cartoid artery. Thus, a conventional substantially occluding stent-graft solution for treating the aneurysm as might be suitable for treating aneurysms elsewhere in the human body, is not appropriate as this would block flow to the perforator vessels and hence cause substantial irreparable damage to a patient.

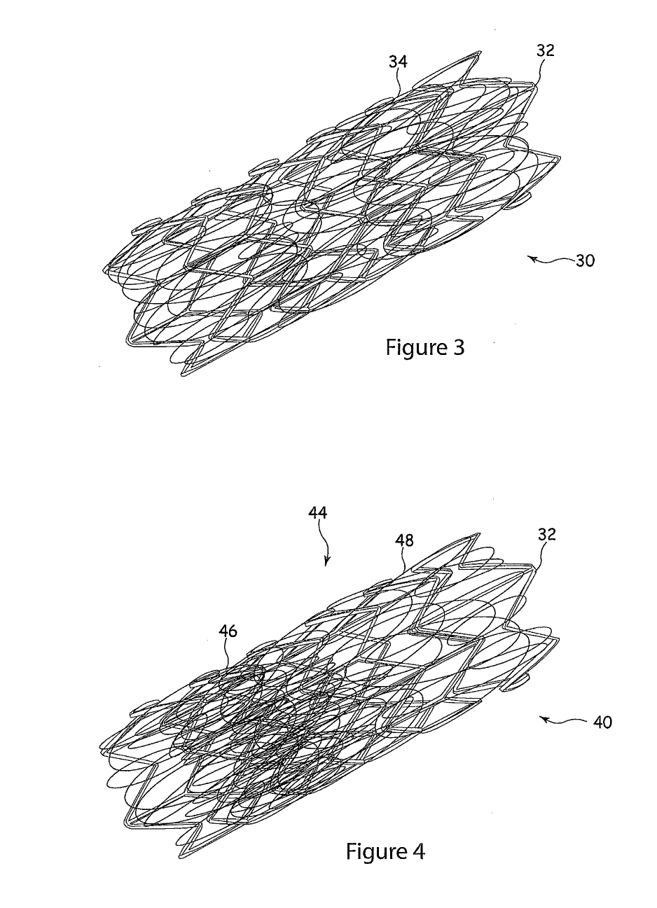

FIGS. 3 to 6 depict various embodiments of coated stent suitable for treating cartoid artery aneurysms. It is envisaged that these embodiments can provide effective treatment of an aneurysm by reducing the flow into the aneurysm rather than by providing an occlusion barrier thereto. In particular, it is envisaged that a flow reduction of around 40% or greater can allow for resorbtion of the aneurysm. The advantage of this solution is that by avoiding an occluding barrier, flow to any perforator vessels adjacent the aneurysm and covered by the stent would still be able to receive blood flow through the covering itself. It has been found that this is assisted by the suction effect in the perforator vessels caused by their blood drain.

Thus, the provision of a permeable covering can provide a stent structure suitable for treating cerebral aneurysms. Moreover, a fibrous covering of the nature disclosed herein does not add noticeable stiffness or volume to the stent, which is particularly important in the case of cartoid arteries.

Referring now to FIG. 3, there is shown an embodiment of covered stent 30 which includes a Z-stent or other stent 32, of conventional form, and a covering 34 of fibrous material adhered to the stent structure. The fibrous material made be made from any suitable polymeric material such as a spin-coatable polymeric material, specific examples being polyurethane, PTFE, polyester, polyurethane, polyethylene and silicon. The preferred embodiment has fibres of a medical grade, aliphatic, polyester based thermoplastic polyurethane such as Tecophilic®. In some instances, the fibres may be biodegradable, in which case they may be made from collagen, polyglycolic acid, polyactive acid, amongst others. It is envisaged that some embodiments could usefully be formed from a fibrous biologically active material, such as collagen or SIS, which contains collagen and growth factors. Such biologically active fibres are enzymatically reduced, allowing rapid recanalization of occluded side branches with adequate flow, while effectively blocking and coagulating aneurisms with no outlet flow. In some instances, a mixture of a biologically active material and a polymer material might be used.

The covering 34 preferably provides a maximum coverage of 70% of the surface area of the underlying stent, preferably a maximum of 50%. The average diameter of the fibres and the thickness of the covering layer 34 is preferably such that they provide a 40% or greater reduction into the cerebral aneurysm. It is envisaged that some implementations have a permeability which reduces flow into an aneurism by between 30 to 60 percent.

In FIG. 3 the fibres of the cover 34 are substantially uniform across all of or substantially all of the underlying stent 32. This therefore provides a structure with a substantially even permeability across substantially the entirety of the surface of the device 30.

FIG. 4 shows another embodiment of stent structure 40 provided with an underlying stent 32 equivalent to that of the embodiment of FIG. 3. On the other hand, in this embodiment, the cover 44, while also formed of a layer of randomly oriented fibres as with the cover 34 of FIG. 3, has areas of different fibre density or numbers so as to provide areas with different permeabilities. In the example shown in FIG. 4, there is shown an area 46 which has more fibres, achieved by the covering in this area being thicker or by the fibres being arranged more densely, and an area 48 with a lower density of fibres. Thus, the permeability in area 46 will be less than the permeability of the area 48.

The advantage of this embodiment of FIG. 4 is that the stent structure can be more appropriately suited to a particular medical application, for example so as to treat an aneurysm in an area of an inner cartoid artery which has a multitude of perforator vessels and for which it is desired to provide substantial reduction of flow into the aneurysm and little or virtually no reduction of flow into the perforator vessels.

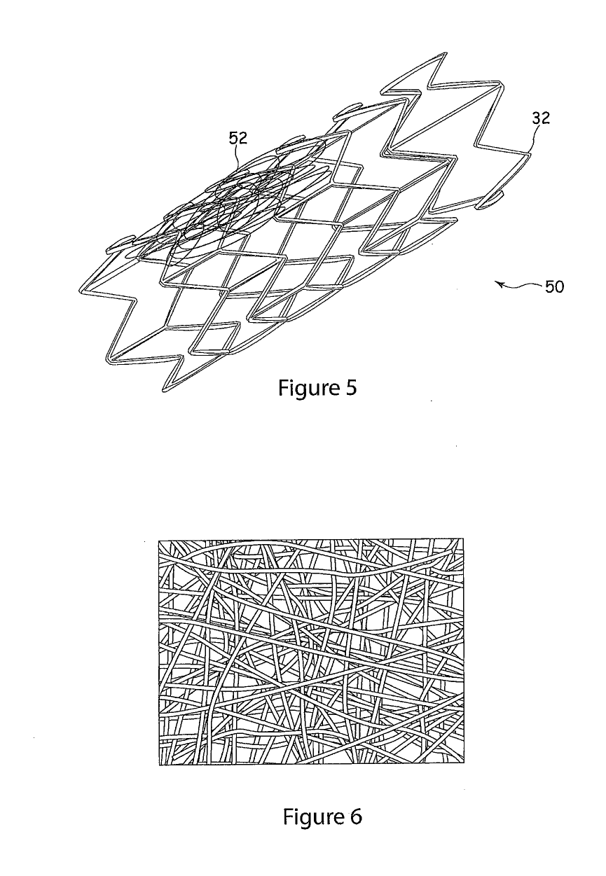

Another embodiment of stent structure is shown in FIG. 5. In this example, the stent structure 50 includes a stent 32 of the type shown in FIGS. 3 and 4 and a covering 52 formed of the same fibres. However, in this embodiment, the covering 52 extends only is one zone of the structure 50, leaving the other parts of the stent 32 uncovered.

In practice, the embodiment of FIG. 5 would be useful for applications in which it is desired to provide flow reduction or occlusion to one a particular area of the stent structure 50, for example to overlie the neck of an aneurysm. Of course, there may be provided more than one covered zone 52, in dependence upon the particular medical condition.

FIG. 6 shows an example of the nature of the fibrous covering of the embodiments of FIGS. 3 to 5. As will be described in further detail below, the manufacturing method provides for the production of randomly oriented fibres overlapping one another with interstitial spaces, as will be evident form FIG. 6. The size and number of spaces and thus the porosity of the fibrous layer can be varied by varying the thickness of the fibres, the density of the fibres as well as the thickness of the fibrous layer. In this manner, a fibrous layer providing the desired or required reduction of flow therethrough can be produced.

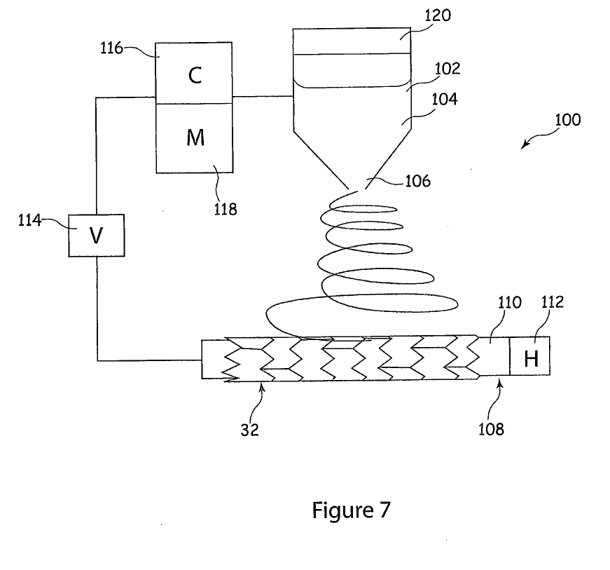

FIG. 7 shows in schematic form an example of apparatus for electro spinning a coating of fibres onto a stent.

The apparatus 100 includes a hopper or syringe 102 containing the polymer or other material 104 to be spun into a coating. The hopper 102 is provided with a nozzle 106 able to release a thin thread of liquid polymer or other composition during the forming process.

A holder 108 includes, in this example, a support mandrel 110 onto which a stent 32 can be fitted. The support mandrel is in this embodiment electrically conductive and is coupled to one terminal of a high voltage power supply 114.

The holder 108 also includes, in this example, a position adjustor 112, for instance one or more electric motors, which is able to rotate the mandrel 11 and thus a stent 32 thereon. The position adjustor may also be able to move the mandrel reciprocally along its longitudinal axis in order to move the longitudinal position of the stent 32 relative to the position of the nozzle 106. In some embodiments there may also be provided a moving device operable to move the mandrel 110 towards and away from the nozzle 106 and in others there may be provided a device for moving the nozzle 106 relative to the mandrel 110. Such devices will be well within the skill and knowledge of the skilled person to implement.

The high voltage supply 114 is also coupled to the hopper 102 so as to impart an electrical charge to the polymer or other material within the hopper, this charge being of the opposite polarity to the charge imparted to the mandrel 110.

There is also provided a control unit 116 as well as a drive unit 118, the latter being operable to drive a pump 120 for pumping the liquid from the hopper 102 through the nozzle 106. The drive unit 116 may also be provided with a device for moving the position and/or orientation of the nozzle 106 in embodiments where this can be moved.

In operation, a stent 32 is placed on the mandrel 108, which is then charged by application of the high voltage from source 114. Polymer 104 is then sprayed through the nozzle 106 by operation of the pump 120. The opposite electrical charge applied to the polymer causes this to accelerate out of the nozzle to form fine fibrils or threads which are attracted to the mandrel. The position of the stent 32 over the mandrel causes all of or at least most of the fibrils or threads to impinge upon the stent 32. As the jet of fibrils or threads from the nozzle is random, the layer deposited on the stent 32 is also random.

By movement of the mandrel 108, under guidance of the controller 116, the nature of the layer of fibrils or threads on the stent 32 can be controlled, that is its density and thickness. For example, a thicker layer can be produced by continuing the process for longer, while a thicker zone can be produced by allowing fibrils to deposit on that zone for longer than other zones of the stent 32.

It is possible to control other aspects of the fibrous coating. For example, the diameter of the fibrils or threads can be varied by changing the density of the polymer solution, by changing the pumping pressure and/or by changing the potential difference between the polymer and the mandrel and hence the acceleration force imparted to the polymer. Similarly, the density of the fibrils can be changed by adjusting the distance between the stent (in practice the mandrel) and the nozzle 106. The characteristics of the fibres can also be determined by selecting different nozzle sizes.

It is preferred that the fibrils or threads are made to impinge upon the stent 32 before these dry. The advantage of this is that the wet fibrils will adhere naturally to the stent as they dry and will adhere to one another at the same time, thus forming a coating which is secured to the stent.

It will be apparent that although the apparatus of FIG. 7 is described with reference to a mandrel which can move, the skilled person will readily appreciate and be able to implement by standard design knowledge a system in which the nozzle 106 may move around a fixed mandrel. For this purpose, the nozzle 106 may be provided at the end of a flexible hose coupled to the hopper 102.

Although the preferred embodiments show a cover which is on the outside of the stent 32, in some embodiments the covering may be on the internal surface of the stent 32. Furthermore, in some instances, a covering may be applied to both of the inner and outer surfaces of the stent.

It is also envisaged that there could be provided a sandwich construction of stent, fibrous covering and braided overlying layer. An additional, outer, fibrous layer may be provided over the braid. Such a device could provide a strong stent, filter or occluder.

Although the described embodiments are directed to a stent, the teachings herein are also applicable to other devices, such as filters and occlusion devices.

1. An endoluminally implantable medical device including a substrate in the form of a substantially open framework and a permeable partial covering of randomly oriented fibrous material located on a part of the substrate.

2. The device according to claim 1, wherein the covering is operable to provide a flow reduction of 40% or greater.

3. The device according to claim 1, wherein the covering has a permeability able to reduce flow therethrough by between 30 to 60 percent of the flow through the device.

4. The device according to claim 1, wherein the fibrous material is or includes a polymer material.

5. The device according to claim 4, wherein the covering is operable to provide a flow reduction of 40% or greater.

6. The device according to claim 4, wherein the covering has a permeability able to reduce flow therethrough by between 30 to 60 percent of the flow through the device.

7. The device according to claim 1, wherein the fibrous material is or includes a biologically active fibrous material.

8. The device according to claim 7, wherein the material is collagen or a collagen based material.

9. The device according to claim 8, wherein the covering is operable to provide a flow reduction of 40% or greater.

10. The device according to claim 8, wherein the covering has a permeability able to reduce flow therethrough by between 30 to 60 percent of the flow through the device.

11. The device according to claim 1, wherein the medical device is a cerebral stent and the covering has a permeability able to reduce flow therethrough by between 30 to 60 percent of the flow through the stent.

12. The device according to claim 11, wherein the covering is operable to provide a flow reduction of 40% or greater.

13. The device according to claim 11, wherein the fibrous material is or includes a polymer material.

14. The device according to claim 11, wherein the fibrous material is or includes a biologically active fibrous material.

15. The device according to claim 14, wherein the material is collagen or a collagen based material.

16. The device according to claim 15, wherein the covering is operable to provide a flow reduction of 40% or greater.

17. The device according to claim 1, wherein the medical device is a vena cava filter.

18. The device according to claim 1, wherein the medical device is an endoluminal occlusion device.

19. A system for providing a coated endoluminal medical device including:
a substrate holder for holding a substrate in the form of a substantially open framework;
a nozzle arrangement directable to the substrate holder;
a source of fluid fibrous material;
a pumping device operable to pump fluid fibrous material through the nozzle arrangement; and
a control unit operable to control the pumping device and the nozzle arrangement;
wherein the control unit is able to apply a variable coating to a substrate.

20. A method of coating endoluminal medical device including the steps of:
holding a substrate in the form of a substantially open framework on a substrate holder;
providing a nozzle arrangement directed at the substrate holder;
pumping fluid fibrous material through the nozzle arrangement; and
wherein the pumping arrangement applies a variable coating to the substrate.
